(19) United States
(12) Patent Application Publication
(10) Pub. No.: US 2016/0089380 A1
(43) Pub. Date: Mar. 31, 2016

(54) PROGESTERONE IMPREGNATED VAGINAL RING/PESSARY

(71) Applicant: YALE UNIVERSITY, New Haven, CT (US)

(72) Inventors: Jonathan Black, New Haven, CT (US); Gary Altwerger, New Haven, CT (US)

(73) Assignee: Yale University

(21) Appl. No.: 14/869,077

(22) Filed: Sep. 29, 2015

Related U.S. Application Data
(60) Provisional application No. 62/056,921, filed on Sep. 29, 2014.

(51) Int. Cl. A61K 31/57; A61F 6/08; A61K 9/00

(52) U.S. Cl. CPC A61K 31/57; A61K 9/0036; A61F 6/08

(57) ABSTRACT

A progesterone impregnated vaginal ring/pessary includes a circumferential outer ring composed of a progesterone impregnated biomaterial, wherein the outer ring has a substantially circular configuration while lying flat with an outer diameter of 60 mm to 70 mm and a thickness of approximately 5 mm. The vaginal ring/pessary also includes a transparent membrane extending along an interior space defined by the outer ring. The transparent membrane is composed of a silicone polymer, has a thickness of 1 mm to 3 mm, and includes a plurality of apertures, which are 4 mm to 5 mm in diameter, circumferentially spaced about a perimeter of the transparent membrane.

### CROSS REFERENCE TO RELATED APPLICATION

This application claims the benefit of U.S. Provisional Patent Application Ser. No. 62/056,921, entitled “PROGESTERONE IMPREGNATED RING/PESSARY,” filed Sep. 29, 2014.

### BACKGROUND OF THE INVENTION

1. Field of the Invention

The invention relates to a progesterone impregnated vaginal ring/pessary utilized in reducing contractions of the uterus.

2. Description of the Related Art

Each year, preterm births affect nearly 500,000 babies, or 1 of every 8 babies born in the United States. Preterm delivery is the main cause of neonatal morbidity and mortality in babies born before 37 weeks, and particularly, those babies born before 34 weeks. Such babies are at greater risk of having problems at delivery and in infancy.

Infants born preterm are also at greater risk of dying in their first year of life, and of those infants who survive, there is an increased risk of hospital admissions and adverse outcomes including cerebral palsy and long-term disabilities. Spontaneous preterm birth arises in 5-13% of pregnancies and is a leading cause of perinatal morbidity and mortality. The strongest predictor of preterm birth is a history of preterm birth. As such, women with a history of preterm birth who find themselves pregnant again receive progesterone supplementation.

Progesterone reduces contractions of the uterus and has important roles in maintaining pregnancy. Side effects of progesterone include headaches, breast tenderness, nausea and coughs. When progesterone is given to mothers at risk of preterm delivery, progesterone has been shown to prolong pregnancy and, therefore, have beneficial effects on the fetus. Such beneficial effects include a reduced risk of newborn death and decreased birth-associated complications such as assisted ventilation, necrotizing enterocolitis and admissions to neonatal intensive care unit.

A Cochrane Report showed that progesterone was associated with a reduction in the risk of preterm birth <34 weeks (two studies; 438 women; RR 0.64, 95% CI 0.45 to 0.90), preterm birth at <28 weeks' gestation (two studies; 1115 women; RR 0.59, 95% CI 0.37 to 0.93). Another Cochrane Report showed that progesterone was associated with a statistically significant reduction in the risk of preterm birth <34 weeks (five studies; 602 women; average RR 0.31, 95% CI 0.14 to 0.69), preterm birth <37 weeks (10 studies; 1750 women; average RR 0.55, 95% CI 0.42 to 0.74) and a statistically significant increase in pregnancy prolongation in weeks (one study; 148 women; mean difference (MD) 4.47, 95% CI 2.15 to 6.79).

Conventional cervical cerclage is also used for women with a history of preterm birth and also in a shortened cervix during pregnancy. It can be associated with complications and is not without risk. Cervical pessary may offer a safe and easy alternative to cerclage for the treatment of cervical insufficiency and prevention of preterm birth. Cervical pessary use could prevent preterm birth in a population of appropriately selected at-risk women.

PECEP (Prevention of Preterm Birth Using Cervical Pessary) Randomized Controlled Trial showed spontaneous delivery at <34 weeks of gestation was significantly less frequent in the pessary group than in the expectant management group (6% vs. 27%, odds ratio 0.18, p<0.0001). No serious adverse effects were associated with the use of a cervical pessary. Ludimir et al. In the pessary group the average gestational age at delivery was 31.5+/−6.8 weeks and in the bed rest group it was noted to be 27.5+/−3.4 weeks (P=0.07). Additional trials are ongoing.

### SUMMARY OF THE INVENTION

It is, therefore, an object of the present invention to provide a progesterone impregnated vaginal ring/pessary. The vaginal ring/pessary includes a circumferential outer ring composed of a progesterone impregnated biomaterial. A transparent membrane extends within and fills an interior space defined by the outer ring.

It is also an object of the present invention to provide a vaginal ring/pessary wherein the outer ring has a substantially circular configuration while lying flat in a relaxed configuration.

It is another object of the present invention to provide a vaginal ring/pessary wherein the outer ring has an outer diameter of 60 mm to 70 mm.

It is a further object of the present invention to provide a vaginal ring/pessary wherein the outer ring has a thickness of approximately 5 mm.

It is also an object of the present invention to provide a vaginal ring/pessary wherein the outer ring includes an upper surface and a lower surface, as well as an exterior surface and an interior surface.

It is another object of the present invention to provide a vaginal ring/pessary wherein the transparent membrane is a silicone polymer.

It is a further object of the present invention to provide a vaginal ring/pessary wherein the transparent membrane includes a plurality of apertures.

It is also an object of the present invention to provide a vaginal ring/pessary wherein the apertures are circumferentially spaced about a perimeter of the transparent membrane.

It is another object of the present invention to provide a vaginal ring/pessary wherein the apertures are 4 mm to 5 mm in diameter.

It is a further object of the present invention to provide a vaginal ring/pessary wherein the transparent membrane has a thickness of 1 mm to 3 mm.

It is also an object of the present invention to provide progesterone impregnated vaginal ring/pessary including a circumferential outer ring composed of a progesterone impregnated biomaterial, wherein the outer ring has a substantially circular configuration while lying flat in a relaxed configuration with an outer diameter of 60 mm to 70 mm and a thickness of approximately 5 mm. The vaginal ring/pessary also includes a transparent membrane extending along an interior space defined by the outer ring. The transparent membrane is composed of a silicone polymer, has a thickness of 1 mm to 3 mm, and includes a plurality of apertures, which are 4 mm to 5 mm in diameter, circumferentially spaced about a perimeter of the transparent membrane.

It is another object of the present invention to provide a progesterone impregnated vaginal ring/pessary including a circumferential outer ring composed of a progesterone impregnated biomaterial and an internal secondary ring composed of a progesterone impregnated biomaterial. An annular transparent membrane extends between the outer ring and the internal secondary ring and fills an annular interior space between the outer ring and the internal secondary ring.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a top plan view of a first embodiment of a progesterone impregnated vaginal ring/pessary in accordance with the present invention.

FIG. 2 is a cross-sectional view along the line 2-2 in FIG. 1.

FIG. 3 is a side plan view of the progesterone impregnated vaginal ring/pessary shown in FIG. 1.

FIG. 4 is a top plan view of a second embodiment of a progesterone impregnated vaginal ring/pessary in accordance with the present invention.

FIG. 5 is a cross-sectional view along the line 5-5 in FIG. 4.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

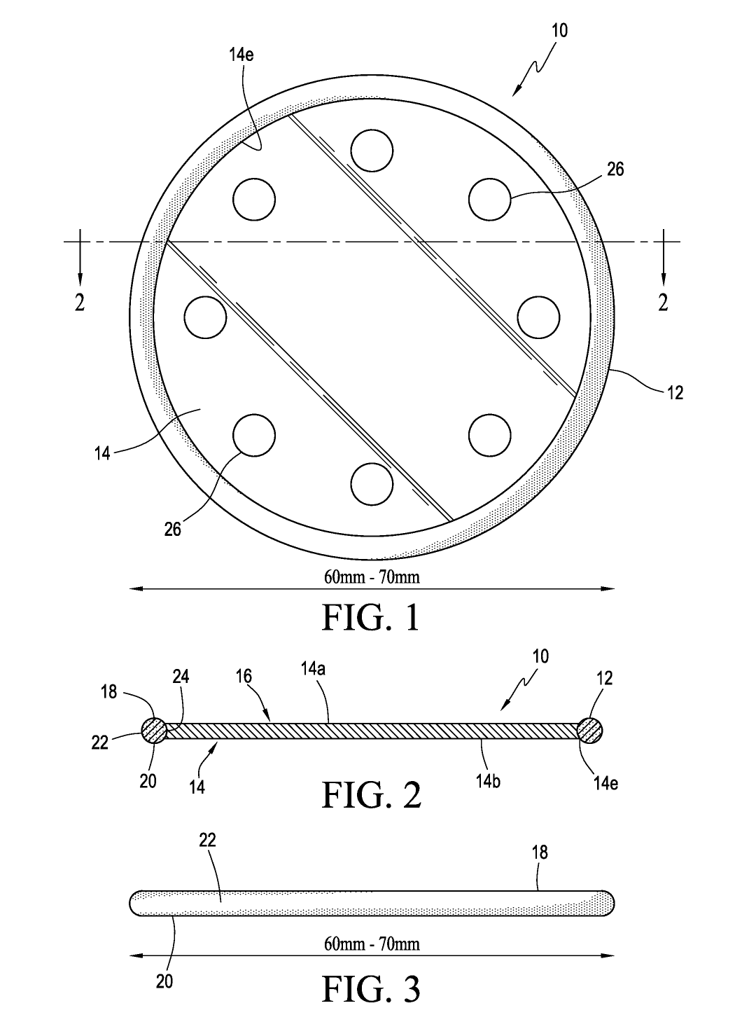

With reference to FIGS. 1, 2 and 3, a first embodiment of a progesterone impregnated vaginal ring/pessary 10 is disclosed. The vaginal ring/pessary 10 includes a circumferential outer ring 12 with a transparent membrane 14 extending within and filling the interior space 16 defined by the outer ring 12. The transparent membrane 14 is clear, and as is explained herein, is constructed to allow for viewing therethrough. As will be appreciated based upon the following disclosure, the present vaginal ring/pessary 10 allows for the delivery of progesterone, while simultaneously allowing for clear observation of the cervix, for example, to monitor cervical dilation.

The outer ring 12 is composed of a progesterone impregnated biomaterial. The outer ring 12 has a substantially circular configuration while lying flat in a relaxed configuration and preferably has an outer diameter of approximately 60 mm to 70 mm. While a preferred embodiment of the present invention utilizes an outer ring that lies within a single plane when in a relaxed state, that is, is of a flat construction, it is contemplated other shapes may be employed without departing from the spirit of the present invention. The outer ring 12 is preferably composed of a flexible and resilient ethylene-vinyl acetate copolymer or silicone polymer, although it is appreciated other flexible and resilient biomaterials may be used without departing from the spirit of the invention. For example, the ethylene-vinyl acetate copolymer disclosed in PCT Publication No. PCT/US2009/040951, entitled “DEVICES THAT INCLUDE ETHYLENE-VINYL ACETATE COPOLYMERS AND METHODS OF MAKING AND USING SAME,” published Oct. 22, 2009, which is incorporated herein by reference. Such a material can achieve varying degrees of a Yong's modulus by having varying degrees of co-polymerization.

As for the cross-sectional shape of the outer ring 12, that is, the shape of a section taken along a plane perpendicular to the plane in which the outer ring 12 lies, it is generally circular (see FIG. 2). The outer ring 12 preferably has a thickness (or cross-sectional diameter in consideration of its circular shape) of approximately 5 mm. It is appreciated the thickness may be varied depending on the material used in the manufacture of the ring and the desired Yong's modulus of the ring. This construct allows relative flexibility for deployment and use in accordance with operating parameters appreciated by those skilled in the field of vaginal rings and pessaries. It is appreciated that the outer ring 12 is shaped and dimensioned to both help in keeping the cervix closed and providing support to the pelvic floor. The vaginal ring/pessary 10 is deployed into the vaginal fornices where the cervix is displaced posteriorly thereby minimizing the direct downward force vector from the uterus and fetus onto the cervix.

As briefly mentioned above, the outer ring 12 defines an interior space 16 in which a transparent membrane 14 is positioned so as to completely fill the interior space. As such, the outer ring 12 includes an upper surface 18 and a lower surface 20, as well as an exterior surface 22 (that is, the surface of the outer ring 12 facing away from the interior space 16) and an interior surface 24 (that is, the surface of the outer ring 12 facing the interior space 16). As such, and as discussed above, the distance between the upper surface 18 and the lower surface is approximately 5 mm and the distance between the exterior surface 22 and the interior surface 24 is approximately 5 mm. It is appreciated that given the circular cross-sectional configuration of the outer ring 12, the terms upper, lower, exterior and interior are relative s.

The circumferential edge 14e of the transparent membrane 14 is secured to the interior surface 24 of the outer ring 12 at a position such that the plane in which the transparent member 14 lies substantially bisects the outer ring 12. In particular, the plane in which the transparent membrane 14 lies is considered to divide the upper surface 18 of the outer ring 12 from the lower surface 20 of the outer ring 12, and the transparent member 14 therefore includes an upper surface 14a along the upper surface 18 of the outer ring 12 and a lower surface 14b along the lower surface 20 of the outer ring 12. In accordance with a preferred embodiment, the transparent membrane 14 is composed of a silicone polymer and has a thickness of approximately 1 mm to 3 mm, although it is contemplated materials such as the ethylene-vinyl acetate copolymer discussed above with regard to the outer ring could be used (with the understanding the Young's modulus of the transparent membrane would be lower when compared to the outer ring). The transparent membrane 14 has a diametric length of approximately 55 mm to 65 mm when measured diametrically across the transparent membrane 14 when the outer ring 12 is in its flat and relaxed state.

The transparent membrane 14 also includes a plurality of apertures 26 extending between the upper surface 14a and the lower surface 14b of the transparent membrane 14. The apertures 26 provide for the passage of bodily fluids as is explained below. The apertures 26 are circumferentially spaced about the perimeter of the transparent membrane 14 at positions adjacent the interior surface 24 of the circumferential outer ring 12. The apertures 26 are preferably approximately 4 mm to 5 mm in diameter and there are preferably 8 apertures spaced about the perimeter of the transparent membrane 14 as shown with reference to FIG. 2,

As mentioned above, the transparent membrane 14 is clear for viewing therethrough and offers protection as will be described below. In particular, the transparent membrane 14 allows a medical practitioner, who is doing a vaginal! speculum exam while the present progesterone impregnated vaginal ring/pessary 10 is deployed, to clearly see cervical dilation. As a result, the medical practitioner may make this visual observation without resorting to a digital exam or additional manipulation. It is appreciated both digital examination and additional manipulation may introduce additional bacteria into the vagina and thus predispose the pregnancy to preterm birth even more so than it already was and the avoidance of such procedures will assist in delaying birth and avoiding preterm birth.

In addition to providing a means for clearly viewing cervical dilation, and as discussed in the background section, the incorporation of progesterone within the outer ring 12 allows for delivery of progesterone to the patient. It is known that progesterone helps maintain a pregnancy. As such, and in accordance with a preferred embodiment of the present invention, the outer ring 12 allows for the controlled release of progesterone at a rate of 200 mcg every night thus eliminating the need for digital placement and again decreasing the likelihood of introducing foreign bacteria.

Further, the present vaginal ring/pessary 10 is sufficiently stiff to support the cervix so as to alleviate potential cervical incompetence. It is appreciated that the present vaginal ring/pessary 10 will be self-introduced by the patient or by the healthcare provider if the patient is uncomfortable doing so herself. In particular, it is appreciated cervical incompetence may be treated with an Arabin pessary. However, the Arabin pessary is useful when there is a measurable cervix, or, a cervix that can go through the opening of the Arabin pessary. Unfortunately, there are instances where the cervix is too short for it to go through an Arabin pessary, or similar pessary, and in such instances the present vaginal ring/pessary 10 may be positioned adjacent the cervix so as to provide support thereof and alleviate potential cervical incompetence. Additionally with the Arabin pessary it is difficult to visualize the cervix during frequent diagnostic examinations to assess the progression of cervical shortening and/or dilation. Still further, the provision of the apertures 26 within the transparent membrane 14 allows for the discharge of any vaginal secretions. As such, any vaginal discharge is permitted to escape and will not build up.

In accordance with the present invention the vaginal ring/pessary is delivered using conventional techniques. For example, the vaginal ring/pessary is inserted into the vagina, as high as possible, using a pessary applicator as is well known to those skilled in the art.

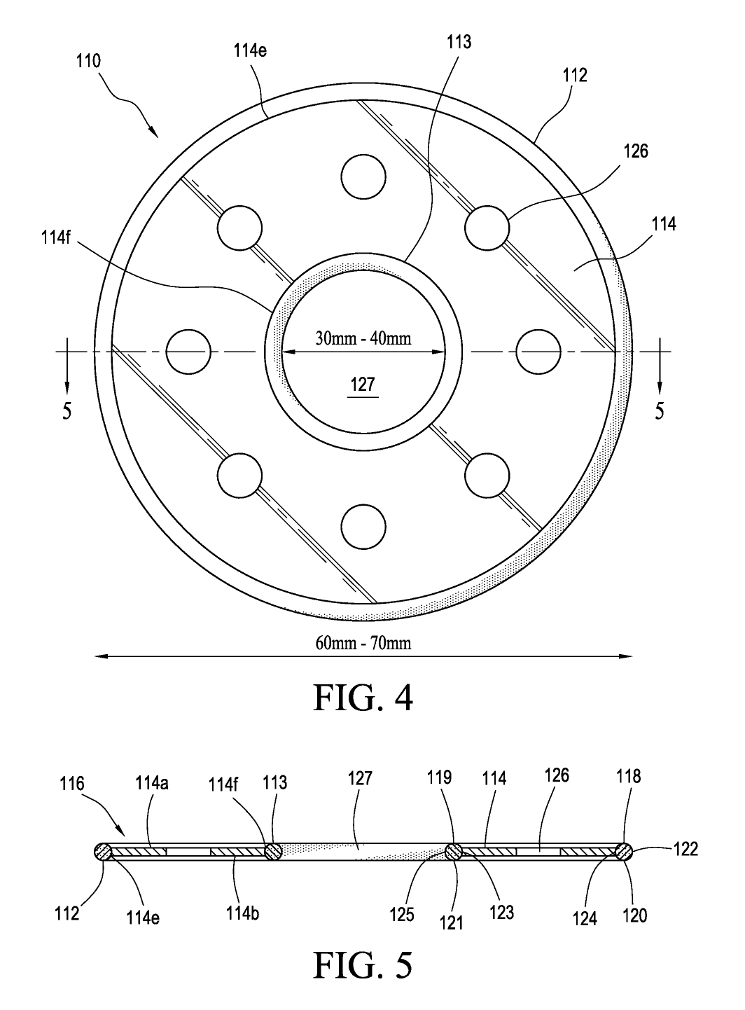

Further to the embodiment disclosed above, the construction of the vaginal ring/pessary may be varied with an internal secondary ring 113 that fits inside of the larger outer ring 112 and an annular transparent membrane 114 that extends between the outer ring and the internal secondary ring 113. In particular and with reference to FIGS. 4 and 5, the vaginal ring/pessary 110 includes a flexible and resilient circumferential outer ring 112 and a flexible and resilient internal secondary ring 114. Both the circumferential outer ring 112 and the internal secondary ring 113 lie in the same plane. The annular transparent membrane 114 extends between the circumferential outer ring 112 and the internal secondary ring 113 and fills the annular interior space 116 between the circumferential outer ring 112 and the internal secondary ring 113. As with the prior embodiment, the vaginal ring/pessary 110 allows for the delivery of progesterone, while simultaneously allowing for clear observation of the cervix, for example, to monitor cervical dilation. As such, the materials disclosed above with reference to the embodiment of FIGS. 1 to 3 are utilized in the construction of this embodiment.

The outer ring 112 is composed of a progesterone impregnated biomaterial. The outer ring 112 has a substantially circular configuration while lying flat in a relaxed configuration and preferably has an outer diameter of approximately 60 mm to 70 mm. As for the cross-sectional shape of the outer ring 112 it is generally circular (see FIG. 5) and has a thickness (or cross-sectional diameter in consideration of its circular shape) of approximately 5 mm. The internal secondary ring 113 also is composed of a progesterone impregnated biomaterial. The internal secondary ring 113 also has a substantially circular configuration while lying flat in a relaxed configuration and preferably has an outer diameter of approximately 30 mm to 40 mm. The internal secondary ring 113 is of a generally circular cross-sectional shape (see FIG. 5) and has a thickness (or cross-sectional diameter in consideration of its circular shape) of approximately 5 mm.

As briefly mentioned above, the outer ring 112 and an internal secondary ring 113 lie in the same plane, and an annular interior space 116 is defined therebetween. The internal secondary ring 113 is positioned in the radial center of the outer ring 112, and the internal secondary ring 113 and the outer ring 112 therefore share the same center point. The transparent membrane 114 is positioned so as to completely fill the annular interior space 116 and therefore is of an annular shape. The outer ring 112 includes an upper surface 118 and a lower surface 120, as well as an exterior surface 122 (that is, the surface of the outer ring 112 facing away from the annular interior space 116) and an interior surface 124 (that is, the surface of the outer ring 112 facing the annular interior space 116 and the interior surface 123 of the internal secondary ring 113). The internal secondary ring 113 includes an upper surface 119 and a lower surface 121, as well as an interior surface 123 (that is, the surface of the internal secondary ring 113 facing the annular interior space 116 and the interior surface of the of the outer ring 112) and a central surface 125 (that is, the surface of the internal secondary ring 113 facing away from the annular interior space 116 and toward the center of the vaginal ring/pessary 110).

The circumferential external and internal edges 114e, 114f of the annular transparent membrane 114 are respectively secured to the interior surface 124 of the outer ring 112 and the interior surface 123 of the internal secondary ring 113 at positions such that the plane in which the transparent member 114 lies substantially bisects the outer ring 112 and the internal secondary ring 113. In particular, the plane in which the transparent membrane 114 lies is considered to divide the upper surfaces 118, 119 of the outer ring 112 and the internal secondary ring 113 from the lower surfaces 120, 121 of the outer ring 12 and the internal secondary ring 113. The transparent member 114 therefore includes an upper surface 114a along the upper surfaces 118, 119 of the outer ring 112 and the internal secondary ring 113 and a lower surface 114b along the lower surfaces 120, 121 of the outer ring 12 and the internal secondary ring 113. As with the prior embodiment, the annular transparent membrane 114 is composed of a silicone polymer (or ethylene-vinyl acetate copolymer as discussed above) and has a thickness of approximately 1 mm to 3 mm. The annular transparent membrane also has a radial length of approximately 15 mm to 35 mm from the external edge 114e to the internal edge 114f when measured radially when the outer ring 112 and the internal secondary ring 113 are in their relaxed and flat states.

The annular transparent membrane 114 also includes a plurality of apertures 126 extending between the upper surface 114a and the lower surface 114b of the annular transparent membrane 114. The apertures 126 provide for the passage of bodily fluids as is explained above. The apertures 126 are circumferentially spaced about the perimeter of the annular transparent membrane 114 at positions between the interior surface 124 of the outer ring 112 and the interior surface 123 of the internal secondary ring 113. The apertures 26 are preferably approximately 4 mm to 5 mm in diameter and there are preferably 8 apertures spaced about the perimeter of the annular transparent membrane 11.4 as shown with reference to FIG. 4. In addition to the apertures 126, the center of the internal secondary ring 113 is open providing an additional aperture 127 defined by the central surface 125 and extending through the vaginal ring/pessary 110.

While the preferred embodiments have been shown and described, it will be understood that there is no intent to limit the invention by such disclosure, but rather, is intended to cover all modifications and alternate constructions falling within the spirit and scope of the invention.

1. A progesterone impregnated vaginal ring/pessary, comprising:
a circumferential outer ring composed of a progesterone impregnated biomaterial;
a transparent membrane extending within and filling an interior space defined by the outer ring.

2. The progesterone impregnated vaginal ring/pessary according to claim 1, wherein the outer ring has a substantially circular configuration while lying flat in a relaxed configuration.

3. The progesterone impregnated vaginal ring/pessary according to claim 2, wherein the outer ring has an outer diameter of 60 mm to 70 mm.

4. The progesterone impregnated vaginal ring/pessary according to claim 2 wherein the outer ring has a thickness of approximately 5 mm.

5. The progesterone impregnated vaginal ring/pessary according to claim 1, wherein the outer ring includes an upper surface and a lower surface, as well as an exterior surface and an interior surface.

6. The progesterone impregnated vaginal ring/pessary according to claim 1, wherein the transparent membrane is a silicone polymer.

7. The progesterone impregnated vaginal ring/pessary according to claim 1, wherein the transparent membrane includes a plurality of apertures.

8. The progesterone impregnated vaginal ring/pessary according to claim 7, wherein the apertures are circumferentially spaced about a perimeter of the transparent membrane.

9. The progesterone impregnated vaginal ring/pessary according to claim 7, wherein the apertures are 4 mm to 5 mm in diameter.

10. The progesterone impregnated vaginal ring/pessary according to claim wherein the transparent membrane has a thickness of 1 mm to 3 mm.

11. A progesterone impregnated vaginal ring/pessary, comprising:
a circumferential outer ring composed of a progesterone impregnated biomaterial, the outer ring having a substantially circular configuration while lying flat with an outer diameter of 60 mm to 70 mm and a thickness of approximately 5 mm;
a transparent membrane extending along an interior space defined by the outer ring, the transparent membrane being composed of a silicone polymer, having a thickness of 1 mm to 3 mm, and including a plurality of apertures circumferentially spaced about a perimeter of the transparent membrane, wherein the apertures are 4 mm to 5 mm in diameter.

12. A progesterone impregnated vaginal ring/pessary, comprising:
a circumferential outer ring composed of a progesterone impregnated biomaterial;
an internal secondary ring composed of a progesterone impregnated biomaterial;
an annular transparent membrane extending between the outer ring and the internal secondary ring and filling an annular interior space between the outer ring and the internal secondary ring.
